(19) United States
(12) Patent Application Publication
(10) Pub. No.: US 2005/0228244 A1
(43) Pub. Date: Oct. 13, 2005

(54) SMALL-SCALE, VITAL-SIGNS MONITORING DEVICE, SYSTEM AND METHOD

(75) Inventor: Matthew John Banet, Del Mar, CA (US)

Correspondence Address: MATTHEW J. BANET, 6540 LUSK BLVD., C200, SAN DIEGO, CA, 92121, US

(73) Assignee: TRIAGE WIRELESS, INC., San Diego, CA (US)

(21) Appl. No.: 10/907,440

(22) Filed: Mar. 31, 2005

Related U.S. Application Data
(63) Continuation of application No. 10/709,014, filed on Apr. 7, 2004.

(51) Int. Cl. A61B 5/00; A61B 5/02

(52) U.S. Cl. 600/301; 600/323; 600/485; 128/903; 600/316; 128/904

(57) ABSTRACT

The invention provides a monitoring device featuring: 1) a housing having a first surface; 2) a sensor pad, positioned on the first surface, that includes a first LED emitting red light, a second LED emitting infrared light, and a photodetector; 3) a data-processing circuit that analyzes a signal from the photodetector to generate a blood pressure value; and 4) means for transmitting the blood pressure value to an external device.

### CROSS REFERENCES TO RELATED APPLICATION

This application is a continuation-in-part application of U.S. patent application Ser. No. 10/709,014, filed Apr. 7, 2004.

### BACKGROUND OF THE INVENTION

The present invention relates to medical devices for monitoring vital signs such as heart rate, pulse oximetry, and blood pressure.

### DESCRIPTION OF THE RELATED ART

Pulse oximeters are medical devices featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ˜630-670 nm) and infrared (λ˜800-1200 nm) wavelengths. The optical module also features a photodetector that detects radiation transmitted or reflected by an underlying artery. Typically the red and infrared LEDs sequentially emit radiation that is partially absorbed by blood flowing in the artery. The photodetector is synchronized with the LEDs to detect transmitted or reflected radiation. In response, the photodetector generates a separate radiation-induced signal for each wavelength. The signal, called a plethysmograph, is an optical waveform that varies in a time-dependent manner as each heartbeat varies the volume of arterial blood, and hence the amount of transmitted or reflected radiation. A microprocessor in the pulse oximeter processes the relative absorption of red and infrared radiation to determine the oxygen saturation in the patient's blood. A number between 94%-100% is considered normal, while a value below 85% typically indicates the patient requires hospitalization. In addition, the microprocessor analyzes time-dependent features in the plethysmograph to determine the patient's heart rate.

Pulse oximeters work best when the appendage they attach to (e.g., a finger) is at rest. If the finger is moving, for example, the light source and photodetector within the optical module typically move relative to the hand. This generates ‘noise’ in the plethysmograph, which in turn can lead to motion-related artifacts in data describing pulse oximetry and heart rate. Ultimately this reduces the accuracy of the measurement. A non-invasive medical device called a sphygmomanometer measures a patient's blood pressure using an inflatable cuff and a sensor (e.g., a stethoscope) that detects blood flow by listening for sounds called the Korotkoff sounds. During a measurement, a medical professional typically places the cuff around the patient's arm and inflates it to a pressure that exceeds the systolic blood pressure. The medical professional then incrementally reduces pressure in the cuff while listening for flowing blood with the stethoscope. The pressure value at which blood first begins to flow past the deflating cuff, indicated by a Korotkoff sound, is the systolic pressure. The stethoscope monitors this pressure by detecting strong, periodic acoustic ‘beats’ or ‘taps’ indicating that the blood is flowing past the cuff (i.e., the systolic pressure barely exceeds the cuff pressure). The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow (i.e., the diastolic pressure barely exceeds the cuff pressure).

Data indicating blood pressure are most accurately measured during a patient's appointment with a medical professional, such as a doctor or a nurse. Once measured, the medical professional manually records these data in either a written or electronic file. Appointments typically take place a few times each year. Unfortunately, about 20% of all patients experience ‘white coat syndrome’ where anxiety during the appointment affects the blood pressure that is measured. White coat syndrome, for example, can elevate a patient's heart rate and blood pressure; this, in turn, can lead to an inaccurate diagnoses. Various methods have been disclosed for using pulse oximeters to obtain arterial blood pressure values for a patient. One such method is disclosed in U.S. Pat. No. 5,140,990 to Jones et al., for a ‘Method Of Measuring Blood Pressure With a Photoplethysmograph’. The '990 patent discloses using a pulse oximeter with a calibrated auxiliary blood pressure to generate a constant that is specific to a patient's blood pressure. Another method for using a pulse oximeter to measure blood pressure is disclosed in U.S. Pat. No. 6,616,613 to Goodman for a ‘Physiological Signal Monitoring System’. The '613 patent discloses processing a pulse oximetry signal in combination with information from a calibrating device to determine a patient's blood pressure.

### SUMMARY OF THE INVENTION

In one aspect, the invention provides a monitoring device featuring: 1) a housing having a first surface; 2) a sensor pad, positioned on the first surface, that includes a first LED emitting red light, a second LED emitting infrared light, and a photodetector; 3) a data-processing circuit that analyzes a signal from the photodetector to generate a blood pressure value; and 4) means for transmitting the blood pressure value to an external device.

In another aspect, the invention provides a system for monitoring the health of a user, the system comprising: 1) the above-mentioned monitoring device; 2) means for measuring the distance traveled by the user for a predetermined time period in order to generate a distance value; 3) a microprocessor capable of analyzing a signal from the monitoring device to generate a plurality of vital sign values; 4) means for measuring a real-time blood glucose level; 5) means for transmitting the plurality of vital sign values, the distance value, and the real-time blood glucose value to a network; 6) a weight scale featuring means for weighing the user to generate a real-time weight value and means for transmitting the weight value to a network; and 7) an off-site computer system configured to receive and display information transmitted over the network.

The invention has many advantages, particularly in providing a small-scale, low-cost medical device that rapidly measures health-related indicators such as blood pressure, heart rate, and blood oxygen content. The device also integrates with an external glucometer and scale through a connection that is either wired (e.g. serial) or wireless (e.g., Bluetooth, 802.15.4, part-15 radio). The device can also include internal circuitry to measure other indicators, such as a pedometer for measuring steps and calories burned, or a GPS system for measuring total distance traveled.

The device makes blood pressure measurements without using a cuff in a matter of seconds, meaning patients can easily monitor this property with minimal discomfort. Ultimately this allows patients to measure their vital signs throughout the day (e.g., while at work), thereby generating a complete set of information, rather than just a single, isolated measurement. Physicians can use this information to diagnose a wide variety of conditions, particularly hypertension and its many related diseases.

The monitor combines all the benefits of conventional blood-pressure measuring devices without any of the obvious drawbacks (e.g., restrictive, uncomfortable cuffs). Its measurement, made with an optical ‘pad sensor’, is basically unobtrusive to the patient, and thus alleviates conditions, such as a poorly fitting cuff, that can erroneously affect a blood-pressure measurement.

The device additionally includes a simple wired or wireless interface that sends vital-sign information to a personal computer. For example, the device can include a Universal Serial Bus (USB) connector that connects to the computer's back panel. Once a measurement is made, the device stores it on an on-board memory and then sends the information through the USB port to a software program running on the computer. Alternatively, the device can include a short-range radio interface (based on, e.g., Bluetooth or 802.15.4) that wirelessly sends the information to a matched short-range radio within the computer. The software program running on the computer then analyzes the information to generate statistics on a patient's vital signs (e.g., average values, standard deviation, beat-to-beat variations) that are not available with conventional devices that make only isolated measurements. The computer can then send the information through a wired or wireless connection to a central computer system connected to the Internet. The central computer system can further analyze the information, e.g. display it on an Internet-accessible website. This way medical professionals can characterize a patient's real-time vital signs during their day-to-day activities, rather than rely on an isolated measurement during a medical check-up. For example, by viewing this information, a physician can delineate between patients exhibiting white coat syndrome and patients who truly have high blood pressure. Physicians can determine patients who exhibit high blood pressure throughout their day-to-day activities. In response, the physician can prescribe medication and then monitor how this affects the patient's blood pressure.

These and other advantages of the invention will be apparent from the following detailed description and from the claims.

### BRIEF DESCRIPTION OF THE DRAWINGS

FIG. 1A is a semi-schematic view of a portable, small-scale monitor that measures blood pressure, pulse oximetry, heart rate, glucose levels, weight, and steps traveled;

FIG. 1B is a semi-schematic view of the monitor of FIG. 1A worn on a patient's belt;

FIG. 2 is a semi-schematic view of the monitor of FIGS. 1A and 1B connecting through a USB port to either a personal computer or personal digital assistant;

FIGS. 3A and 3B are schematic views of an Internet-based system that receives information from the small-scale monitor of FIGS. 1A and 1B through, respectively, a wired or wireless connection; and

FIG. 4 is a schematic diagram of the electrical components of the small-scale monitor of FIGS. 1A and 1B.

### DETAILED DESCRIPTION OF THE INVENTION

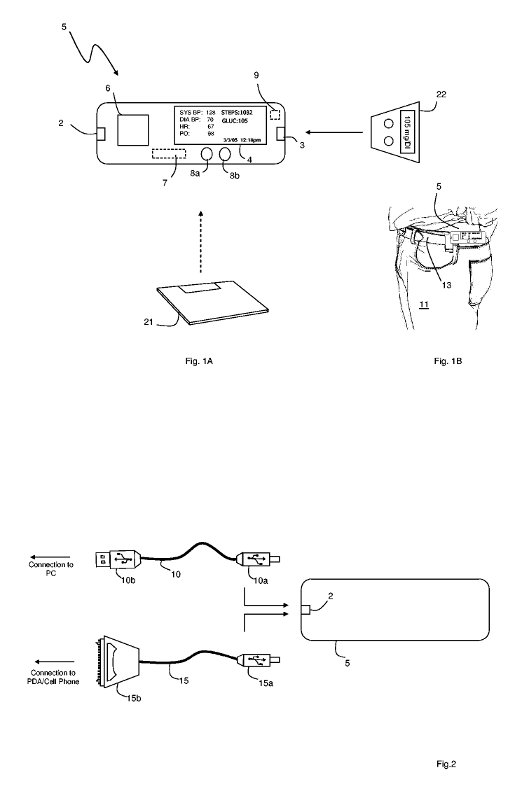

FIGS. 1A and 1B show a portable, small-scale, vital-sign monitor 5 that measures information such as blood pressure, pulse oximetry, heart rate, glucose levels, calories burned, steps traveled, and dietary information from a patient 11. The monitor 5, typically worn on the patient's belt 13, features: i) an integrated, optical ‘pad sensor’ 6 that cufflessly measures blood pressure, pulse oximetry, and heart rate from a patient's finger as described in more detail below; and ii) an integrated pedometer circuit 9 that measures steps and, using an algorithm, calories burned. To receive information from external devices, the monitor 5 also includes: i) a serial connector 3 that connects and downloads information from an external glucometer 22; and ii) a short-range wireless transceiver 7 that receives information such as body weight and percentage of body fat from an external scale 21. The patient views information from a liquid crystal display (LCD) display 4 mounted on the monitor 5, and can interact with the monitor 5 (e.g., reset or reprogram it) using a series of buttons 8a, 8b. The monitor can be used for a variety of applications relating to, e.g., disease management, health maintenance, and medical diagnosis.

Referring to FIG. 2, to transfer information to Internet-accessible devices, the monitor 5 includes a mini USB port 2 that connects to a personal computer through a conventional USB connector 10b terminating a first cable 10. Alternatively, the monitor connects to a personal digital assistant (PDA) through a serial connector 15b terminating a second cable 15. The PDA, for example, can be a conventional wireless device, such as a cellular phone.

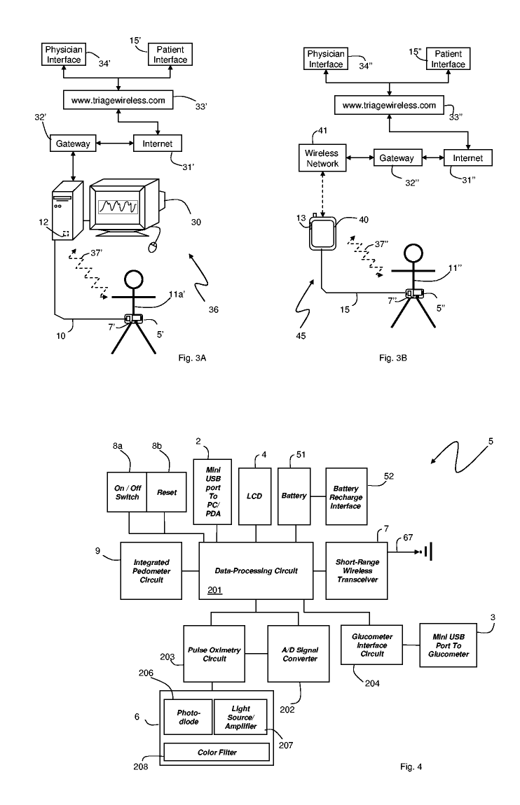

FIGS. 3A and 3B show preferred embodiments of Internet-based systems 36, 45 that operate in concert with the small-scale monitor 5′, 5″ to send information from the patient 11′, 11″ to an Internet-accessible website 33′, 33″. There, a user can access the information using a conventional web browser through a patient interface 15′, 15″ or a physician interface 34′, 34″. Typically the patient interface 15′, 15″ shows information from a single user, whereas the physician interface 34′, 34″ displays information for multiple patients. In both cases, information flows from the monitor 5′, 5″ through a USB cable 10, 15 to an external device (e.g., a personal computer 30 or PDA 40). The personal computer 30 connects to the Internet 31′ through a wired gateway software system 32′, such as an Internet Service Provider. Alternatively, the monitor 5″ wirelessly sends information through a wireless network 41 to a wireless gateway 32″, which then transfers the information to the Internet 31″.

In other embodiments, the small-scale monitor 5′, 5″ transmits patient information using a short-range wireless transceiver 7′, 7″ through a short-range wireless connection 37′, 37″ (e.g., Bluetooth, 802.15.4, part-15) to either the personal computer 30 or PDA 40. For example, the small-scale monitor 5′ can transmit to a matched transceiver 12 within (or connected to) the personal computer 30, or alternatively to a transceiver 13 within the PDA 40. In both cases, the monitor 5 collects and stores information from the patient 11′, 11″, and then transmits this when the monitor 5 roams within range of the personal computer 30 or PDA 40.

During typical operation, the patient 11 uses the monitor 5 for a period of time ranging from a 1-3 months. Typically the patient 111 takes measurements a few times throughout the day, and then uploads the information to the Internet-based systems 36, 45 using a wired or wireless connection. To view patient information sent from the monitor 5, the patient 11 (or other user) accesses the appropriate user interface hosted on the website 33 through the Internet 31.

FIG. 4 shows a preferred embodiment of the electronic components within the monitor 5. A data-processing circuit 201 controls: i) a pulse oximetry circuit 203 connected to an optical pad sensor 6; ii) LCD 4; iii) a glucometer interface circuit 204 that connects to an external glucometer through a mini USB port 3; iv) an integrated pedometer circuit 9; and v) a short-range wireless transceiver 7. During operation, the optical pad sensor 6 generates an optical waveform that the data-processing circuit 201 processes to measure blood pressure, pulse oximetry, and heart rate as described in more detail below. The sensor 6 combines a photodiode 206, color filter 208, and light source/amplifier 207 on a single silicon-based chip. The light source/amplifier 207 typically includes light-emitting diodes that generate both red (λ˜350 nm) and infrared (λ˜1050 nm) radiation. As the heart pumps blood through the patient's finger, blood cells absorb and transmit varying amounts of the red and infrared radiation depending on how much oxygen binds to the cells' hemoglobin. The photodiode 206 detects transmission at both red and infrared wavelengths, and in response generates a radiation-induced current that travels through the sensor 6 to the pulse-oximetry circuit 203. The pulse-oximetry circuit 203 connects to an analog-to-digital signal converter 202, which converts the radiation-induced current into a time-dependent optical waveform. The analog-to-digital signal converter 202 sends the optical waveform to the data-processing circuit 201 that processes it to determine blood pressure, pulse-oximetry, and heart rate, which are then displayed on the LCD 4. Once information is collected, the monitor 5 can send it through a mini USB port 2 to a personal computer 30 or PDA 40, as described with reference to FIGS. 3A, 3B.

In other embodiments, the monitor 5 connects through the mini USB port 3 and glucometer interface circuit to an external glucometer to download blood-glucose levels. The monitor 5 also processes information from an integrated pedometer circuit 9 to measure steps and amount of calories burned.

The monitor 5 includes a short-range wireless transceiver 7 that sends information through an antenna 67 to a matched transceiver embedded in an external device, e.g. a personal computer or PDA. The short-range wireless transceiver 7 can also receive information, such as weight and body-fat percentage, from an external scale. A battery 51 powers all the electrical components within the small-scale monitor 5, and is preferably a metal hydride battery (generating 3-7V) that can be recharged through a battery-recharge interface 52. The battery-recharge interface 52 can receive power through a serial port, e.g. a computer's USB port. Buttons control functions within the monitor such as an on/off switch 8a and a system reset 8b.

To complement measurement of the optical waveform, the pad sensor can also include an electrode that detects an electrical impulse from the patient's skin that is generated each time the patient's heart beats. Following a heartbeat, the electrical impulse travels essentially instantaneously from the patient's heart to the pad sensor, where the electrode detects it to generate an electrical waveform. At a later time, a pressure wave induced by the same heartbeat propagates through the patient's arteries and arrives at the pad sensor, where the light source/amplifier and photodiode detect it as described above to generate the optical waveform. The propagation time of the electrical impulse is independent of blood pressure, whereas the propagation time of the pressure wave depends strongly on pressure, as well as mechanical properties of the patient's arteries (e.g., arterial size, stiffness). The data-processing circuit runs an algorithm that analyzes the time difference (ΔT) between the arrivals of these signals, i.e. the relative occurrence of the optical and electrical waveforms as measured by the pad sensor. Calibrating the measurement (e.g., with a conventional blood pressure cuff) accounts for patient-to-patient variations in arterial properties, and correlates ΔT to both systolic and diastolic blood pressure. This results in a calibration table. During an actual measurement, the calibration source is removed, and the data-processing circuit analyzes ΔT along with other properties of the optical and electrical waveforms and the calibration table to calculate the patient's real-time blood pressure.

Methods for processing optical and electrical waveforms to determine blood pressure without using a cuff are described in the following co-pending patent applications, the entire contents of which are incorporated by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL-SIGN MONITOR FOR ATHLETIC APPLICATIONS (U.S. Ser. No. ______; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); and 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); and PATCH SENSOR FOR MEASURING BLOOD PRESSURE WITHOUT A CUFF (U.S. Ser. No. 10/906,315; filed Feb. 14, 2005).

Still other embodiments are within the scope of the following claims.

1. A monitoring device comprising:
a housing having a first surface;
a sensor pad positioned on the first surface, the sensor pad comprising a first light-emitting diode emitting red light, a second light-emitting diode emitting infrared light, and a photodetector;
a microprocessor capable of analyzing a signal from the photodetector to generate a blood pressure value; and
means for transmitting the blood pressure value to an external device.

2. The monitoring device according to claim 1, wherein the transmitting means is a serial connection.

3. The monitoring device according to claim 1, wherein the serial connection is a USB connection.

4. The monitoring device according to claim 1, wherein the transmitting means is a transceiver that operates a wireless protocol based on Bluetooth™, 802.11a, 802.11b, 802.1g, or 802.15.4.

5. The monitoring device according to claim 1, further comprising an interface to an external scale.

6. The monitoring device according to claim 5, wherein the interface is a wireless interface.

7. A system for monitoring the health of a user, the system comprising:
a monitoring device comprising:
a housing having a first surface;
a sensor pad positioned on the first surface of the housing, the sensor pad comprising a pulse oximetry component;
a pedometer;
a microprocessor capable of analyzing a signal from the pulse oximetry component to generate a real-time blood pressure value of the user of the monitoring device;
means for transmitting the real-time blood pressure value and a distance value from the pedometer to a network; and
an off-site computer system configured to receive and display the blood-pressure information transmitted over the network.

8. The system according to claim 7, wherein the transmitting means of the monitoring device comprises a short-range wireless component that operates a wireless protocol based on Bluetooth™, 802.11a, 802.11b, 802.1g, or 802.15.4.

9. The system according to claim 7 wherein the transmitting means of the monitoring device is a serial connection.

10. The system according to claim 9, wherein the serial connection is a USB connection.

11. The system according to claim 7, further comprising a personal digital assistant that receives the transmission from the transmission means and transmits the blood pressure value and the distance value from the pedometer to the off-site computer system over a wireless network.

12. The system according to claim 111 wherein the personal digital assistant is configured to wirelessly transmit information over a terrestrial wireless network.

13. The system according to claim 7, further comprising a weight scale comprising means for weighing a user and means for transmitting the user's weight to the monitoring device.

14. The system according to claim 13 wherein the transmitting means of the weight scale comprises a short-range wireless component that operates a wireless protocol based on Bluetooth™, 802.11a, 802.11b, 802.1g, or 802.15.4.

15. The system according to claim 13 wherein the transmitting means of the weight scale is a serial connection.

16. The system according to claim 11 wherein the personal digital assistant is configured for two-way messaging over the network between the personal digital assistant and an off-site computer system.

17. The system according to claim 13, further comprising an interface configured to receive dietary information for a user.

18. A system for monitoring the health of a user, the system comprising:
a monitoring device comprising:
a pulse oximetry component;
means for measuring the distance traveled by the user for a predetermined time period in order to generate a distance value;
a microprocessor capable of analyzing a signal from the pulse oximetry component to generate a plurality of vital sign values of the user;
means for measuring a real-time blood glucose level of the user;
means for transmitting the plurality of vital sign values, the distance value, and the real-time blood glucose value to a network;
a weight scale comprising means for weighing the user to generate a real-time weight value and means for transmitting the user's weight value to a network; and
an off-site computer system configured to receive and display information transmitted over the network.
